(19) United States
(12) Patent Application Publication
(10) Pub. No.: US 2007/0141703 A1
(43) Pub. Date: Jun. 21, 2007

(54) Methods for producing blood products from pluripotent cells in cell culture

(76) Inventors: Edouard Guy Stanley, Ascot Vale (AU); Andrew George Elefanty, Surrey Hills (AU); Elizabeth Siewsun Stadler, Surrey Hills (AU); Stephen Anthony Livesey, Victoria (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/579,712

(22) PCT Filed: Nov. 19, 2004
(86) PCT No.: PCT/AU04/01593

§ 371 Date: Jan. 3, 2007

Related U.S. Application Data
(60) Provisional application No. 60/523,249, filed on Nov. 19, 2003.

(51) Int. Cl. C12N 5/08

(52) U.S. Cl. 435/372

(57) ABSTRACT

The present invention provides methods for in vitro production of clinically useful quantities of differentiated human blood cells. In various embodiments of the present invention, immortal pluripotent cells are used to produce differentiated blood cell populations using a cell production device. In a specific embodiment, the device is a sequential series of bioreactors utilizing growth media containing specific combinations of maintenance-, proliferation- or differentiation-promoting factors that maintain, expand and promote the maturation and differentiation of the desired cell types. The immortal pluripotent cells can optionally be genetically modified so as to remove histcompatibility or blood group antigens.

### FIELD OF THE INVENTION

This invention relates generally to the in vitro production of clinically useful quantities of mature blood cells and blood products from immortal human stem cell populations, e.g., human embryonic stem cells.

### BACKGROUND OF THE INVENTION

The availability of transfusible blood products is inadequate to meet current needs. The high incidence of blood borne diseases such as HIV and hepatitis in some countries severely limits the number of available donors and increases the risk of contracting an infection from an autologous blood transfusion. Moreover, even with improvements in the accuracy of blood typing and cross-matching, there continue to be risks associated with blood transfusion including febrile or urticarial reactions and non-fatal or fatal hemolytic reactions.

Human embryonic stem cells (hESCs) are derived from the morula or inner cell mass of a blastocyst-stage human embryo, and, under certain in vitro culture conditions are capable of undergoing an unlimited number of cell divisions without differentiating while maintaining a stable, diploid complement of chromosomes. hESCs are pluripotent and, under various culture regimes, can give rise to any of the differentiated cell types derived from the three primary germ layers of the embryo (the endoderm, mesoderm and ectoderm). The path of differentiation from an hESC to a fully-differentiated cell involves a series of steps resulting in a series of cell intermediates. As the differentiation process advances, it leads to a progressive diminution of the differentiation potential of each resulting cell.

A robust method for generating most differentiated cell types from hESCs is through the creation of embryoid bodies (that is, tissue like spheroids of cellular aggregates derived from one or a number of hESCs.) Methods practiced in the art of producing embryoid bodies include hanging drop, liquid suspension, and methylcellulose cultures (see e.g., Dang et al, Stem Cells 22:275 (2004) and Dang et al, Biotechnology and Bioengineering 74:442 (2002)). Each of these methods relies on the spontaneous aggregation of hESCs in a cell culture medium at the initiation of differentiation.

hESCs can, under certain specific culture conditions and through a series of intermediates, give rise to mature hematopoietic cells. Mature hematopoietic cells comprise lymphoid and myeloid cells. The lymphoid lineages, including B cells and T cells, provide for the production of antibodies, regulation of the cellular immune system, and detection of agents foreign to the host. The myeloid lineages, which include monocytes, dendritic cells, granulocytes, megakaryocytes as well as other cell types, monitors for the presence of foreign bodies, provides protection against neoplastic cells, scavenges foreign materials, and produces platelets. The erythroid lineage produces red blood cells, which carry oxygen.

There are many uses for blood cells and blood products. For example, platelets are used clinically in prophylaxis and treatment of thrombocytopenic hemorrhage as well as provide a source of physiologically relevant factors. Red blood cells are transfused to support the transport of oxygen in situations of hemorrhage or anemia. Specific lymphocytes find application in the treatment of various immunodeficiency diseases, for example, where the lymphocyte is specifically sensitized to an epitope of an antigen. Blood products also may be used for rescue from high dose cancer chemotherapy or for many other purposes.

Several attempts have been made to address the need for transfusible blood products. For example, native hemoglobin has been chemically modified by various methods in an attempt to create a blood substitute, but thus far such products suffer from a variety of shortcomings, including nephrotoxicity, excessive O₂ affinity, a short half-life, rapid dimerization and excretion, and insufficient plasma concentration (see, e.g., Skolnick, J. Amer. Med. Assoc. 268:697 (1992); Vigerou et al., Bull. Acad. Natl. Med., 174:947 (1990)). Alternatively, human hemoglobin has been packaged in liposomes for administration as neo-erythrocytes, but such products are difficult to sterilize (particularly against viruses such as HIV), exhibit a short half-life because they are rapidly cleared by the reticuloendothelial system, and suppress the immune system significantly, thereby predisposing recipients to an increased infection rate (Djordjerich et al, Crit. Rev. Ther. Carrier Syst., 6:131 (1989)). In addition, perfluorochemicals have been tested as hemoglobin substitutes, but these perfluorocarbons contain a potentially toxic surfactant (Pluronic F-68), they must be stored frozen, and, due to their insolubility, require emulsification. Moreover, these fluids require oxygen-enriched air for proper oxygen delivery, as well as frequent administration due to a short half-life.

Despite these efforts, an effective and safe blood substitute is still not available. Thus, there is a desire in the art for the in vitro production of clinically useful quantities of mature blood cells and blood products.

### SUMMARY OF THE INVENTION

The present invention provides methods and apparatus to produce clinically useful amounts of natural, mature, differentiated, universally-compatible, or, in some instances, specifically-engineered human blood cells and blood products under conditions such that the major risks from blood-borne infectious agents and transfusion reactions are absent

Immortal pluripotent cells (e.g., hESCs) are cultured in the presence of combinations of maintenance-, proliferation- and growth- and/or maturation-promoting factors, such as cytokines, lymphokines, colony stimulating factors, mitogens, growth factors, and/or other maturation factors so as to produce at will clinically useful quantities of infectious agent-free human blood cells such as erythrocytes, lymphocytes, megakaryocytes and platelets, monocytes, macrophages, dendritic cells, neutrophils, eosinophils and basophils, and plasma, as well as expanded stem cell cultures. The immortal pluripotent cells to be cultured will preferably differentiate into blood group type O, and Rh factor negative (“universal donor cells”).

Populations of single cell species may be produced via this process or alternatively the cell populations may have a number of cell species which may be separated using fractionation technologies which are already commonly used in the processing of blood which has been provided by a blood donor. In a further alternative, the cell populations produced via this process may be used to produce specific proteins or other cellular factors for therapeutic use.

In one aspect, the present invention provides a method, comprising the steps of: culturing a plurality of immortal pluripotent cells in the presence of a cell culture medium under conditions which promote growth; allowing a portion of the cells to grow and differentiate into differentiated human blood cells; and isolating the differentiated human blood cells from the culture.

In another aspect, the present invention provides a method, comprising the steps of: aggregating at least a portion of a plurality of immortal pluripotent cells; culturing the cells in the presence of a cell culture medium under conditions which promote growth; allowing a portion of the cells to grow and differentiate into differentiated human blood cells; and isolating the differentiated human blood cells from the culture.

In general, it is one aspect of this invention to provide a hematopoietic cell production device comprising a sequential series of bioreactors and selection systems. This cell production device will provide a method to:
1) culture immortal pluripotent cells (e.g., hESCs);
2) induce differentiation of the pluripotent cells into transient amplifying (TA) cells; and
3) differentiate TA cells into increasingly-differentiated TA cells and, ultimately, into fully differentiated mature cells from the hematopoietic lineage.

This aspect of the invention may include the aggregation of the pluripotent cell populations prior to step 2).

In a specific embodiment, cells collected in a final step of the system are in a similar concentration as found in native blood. In another specific embodiment, cells collected in a final step contain a desired cell population in a higher concentration than normally found in native blood. In either case, the cells collected in the final step can be provided in pharmaceutically acceptable solution or can be processed using fractionation or separation technologies to provide cells or products which can be provided in pharmaceutically acceptable solution. The cells in the pharmaceutically acceptable carrier are a therapeutic product for delivery via transfusion of the cells to the circulatory system of a patient.

For example, it is one aspect of this invention to provide a sequential series of bioreactors and selection systems for producing clinically useful quantities of universal-donor erythrocytes from universal-donor hESCs for transfusion into, e.g., anemic or thrombocytopenic patients. In other aspects of the invention, different apparatus and methods will be used to generate different species of mature cells of other hematopoietic lineages, including the myeolocytic lineage.

An important aspect of the invention is a starting “culture” of immortal cells that are self renewable over a span of time, preferably at least three months, more preferably at least six months, but cells that are renewable for one year or longer are even more preferable for use as a starting culture. The current invention is described throughout as using hESCs as the starting cell population, but it is also envisaged that other immortal pluripotent cell populations (e.g., subpopulations of hESC that are optimized for hematopoietic differentiation or modified HSC de-differentiated or otherwise modified or treated in a manner to allow self-renewal in culture) could be used. Thus, although the described embodiments use hESCs as the exemplary immortal pluripotent cell type, the invention is also intended to include other immortal cell populations.

It is yet another aspect of the invention to provide a method for the production of genetically-modified immortal pluripotent cells so as to eliminate or modify cell surface antigens, such as human histocompatibility antigens and blood group antigens. These genetically-modified immortal pluripotent cells can then be expanded and differentiated to produce, for example, single species of erythrocytes, platelets, leukocytes and other mature blood cells for transfusion purposes.

Thus, the present invention provides methods, devices and apparatus to produce blood products ex vivo comprising culturing immortal pluripotent cells in a culture bioreactor, optionally aggregating the immortal cells to produce spheroid bodies within a defined size range, exposing the pluripotent cells to culture conditions that produce TA cells or a combination of daughter pluripotent cells and differentiating TA cells; optionally removing the TA cells from the culture reactor; proliferating the TA cells in a proliferation reactor, which in one embodiment is a separate reactor, and in another embodiment is the culture reactor with altered cell culture conditions; and differentiating the TA cells in a differentiation reactor to produce a population of mature blood cells. In addition, one or more selection or filtration steps may be performed after the culturing step, after the removing step, after the proliferating step and/or after the differentiation step. Also, the method may include one to many additional differentiation steps in sequence or alternating with one or more selection steps. Moreover, a preservation and/or packaging step may be performed after the differentiating step or, if desirable, at an earlier stage to preserve a TA cell for later differentiation.

These and other aspects, advantages, and features of the invention will become apparent to those persons skilled in the art upon reading the details of the bioreactor culture system as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 is a flow chart showing the steps of a method according to one embodiment of the present invention.

FIG. 2A shows a dividing scheme for hESCs in one specific embodiment that provides a steady state population of hESCs while generating TA cells (cells in the process of differentiation). FIG. 2B shows a dividing scheme for hESCs that provides for the expansion or amplification of an hESC population.

FIG. 3 is a diagram showing one model of the lineage relationships of adult immune and blood cells at different stages of development, from an hESC (depicted at the left) to cells of increasing differentiation (as seen to the right).

FIG. 4 is a bar graph representing the number of wells in which blood cell populations were found in the experiments described in Examples 1 and 2 for hESC line 1.

FIG. 5 is a bar graph representing the number of wells in which blood cell populations were found in the experiments described in Examples 1 and 2 for hESC line 2.

FIG. 6 shows the effect of cell density on the percentage of wells containing blood cells for hESC line 1, based on experiments described in Example 3.

FIG. 7 shows the effect of cell density on the percentage of wells containing blood cells for hESC line 2, based on experiments described in Example 3.

### DETAILED DESCRIPTION OF THE INVENTION

Before the present bioreactor systems, devices methods are described, it is to be understood that this invention is not limited to particular systems or methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context dearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a bioreactor” includes a plurality of such devices and reference to “a differentiating factor” includes reference to one or more combinations of such factors and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention. For example, additional description of apparatus, methods, cell populations and appropriate factors that could be employed for the methods of expansion and differentiation described herein include those described in U.S. Pat Nos. 5,399,493, 5,472,867, 5,635,386, 5,635,388, 5,646,043, 5,674,750, 5,925,567, 6,403,559, 6,455,306, 6,258,597, and 6,280,718.

Generally, conventional methods of cell culture, stem cell biology, and recombinant DNA techniques within the skill of the art are employed in the present invention. Such techniques are explained fully in the literature, see, e.g., Maniatis, Fritsch & Sambrook, Molecular Cloning: A Laboratory Manual (1982); DNA Cloning: A Practical Approach, Volumes I and II (D. N. Glover, ed. 1985); Animal Cell Culture (R. I. Freshney, ed. 1986); and Embryonic Stem Cells, (Kurstad Turksen, Ed., Humana Press, 2002).

### DEFINITIONS

The terms “blood cells” or “hematopoietic cells” are intended to include erythrocytes (red blood cells), reticulocytes, megakaryocytes and platelets, eosinophils, neutrophils, basophils, monocytes, macrophages, dendritic cells, granulocytes and cells of the lymphoid lineage and the precursor cells of all of these lineages. For the purpose of transfusion into patients, erythrocytes, granulocytes and platelets may be particularly valuable. The phrase “clinically useful quantities (or amounts) of blood cells” is intended to mean quantities of blood cells of whatever type sufficient for transfusion into human patients to treat a clinical condition. In addition the term “transient amplifying cell” or “TA cell” refers to an intermediately differentiated cell—that is, a cell more differentiated than the initial immortal pluripotent cell, yet less differentiated than mature cells of the hematopoietic lineage such as those listed above.

Procurement of hESCs

Human embryonic stem cells (hESCs) may be derived from the inner cell mass of a blastocyst stage human embryo or an established cell line may be used (such as those developed by Thomson and Odorico, Trends Biotechnol. 18:53-57 (2002), namely, H1, H7, H9.1, H9.2, H13 or H14). To generate human ES cell cultures de novo, cells from the inner cell mass are separated from the surrounding trophectoderm by microsurgery or by immunosurgery (which employ antibodies against the trophectoderm that break it down) and are plated in culture dishes containing growth medium supplemented with fetal bovine serum (alternatively, KnockOut Dulbecco's modified minimal essential medium containing basic FGF can be supplemented with Serum Replacer (Life Technologies) and used without serum), usually on feeder layers of mouse embryonic fibroblasts that have been mitotically inactivated to prevent replication. Alternatively, a feeder-free culture system may be employed, such as that reported by Chunhui Xu, Melissa Carpenter and colleagues using Matrigel or laminin as a substrate, basic FGF, and conditioned medium from cultures of mouse embryo fibroblasts (Xu, et al., Keystone Symposia. Pluripotent stem cell biology and applications: Growth of undifferentiated human embryonicstem cells on defined matricies with conditional medium. Poster abstract 133).

Optional Selection of hESC Cells

Once the hESC culture has been established, it can be placed within the first bioreactor (the culture bioreactor) of the present invention for growth, although an optional selection or forced aggregation step may be performed before transferring the initial hESC culture into the bioreactor.

Selection at this juncture and in other steps in the methods according to the present invention can be performed in any way known in the art. The most robust selection method for hESCs and various TA intermediates to date employs the use of cell surface markers specific to a desired cell type (or cell surface markers specific to an undesired cell type when employing negative selection). Cell surface markers are specialized proteins or glycoproteins, often receptors, which have the capability of selectively binding or adhering to other signalling molecules. Cell surface markers differ markedly in their structure and their affinity for ligands. In many cases, a combination of multiple markers is used to identify a particular cell type. The cell surface markers are exploited for selection by using, e.g., fluorescent labelling and fluorescent activated cell sorting (FACS) where an antibody (usually monoclonal) or other ligand that specifically binds to the cell surface marker is directly or indirectly fluorescently labelled and allowed to bind to cells within a heterogeneous cell population. The cells are subjected to light excitation at a wavelength appropriate for the fluorophore, and then cells expressing the cell surface marker detected by the fluorescent antibody are detected and isolated by virtue of their fluorescent profile.

Alternatively, antibodies or other ligands to the cell surface markers can be immobilized to a surface of a culture flask, bead, or other surface (such as the surface of a bioreactor), and the cells to be sorted are exposed to the ligand/ immobilizing surface. Cells that have cell surface receptors to the ligand will bind the ligand becoming immobilized, where cells lacking the cell surface receptor to the ligand will not bind and can eluted or otherwise separated from the bound cells.

Any ligand that is specific for hESCs may be used in order to obtain a homogenous population of hESCs for use in the methods of the present invention. For example, such a ligand may be an antibody to a cell surface marker. In one embodiment of the invention, the ligand is a monoclonal antibody to cell surface marker CD30 (cluster designation 30), a molecule found specifically on hESCs. Other ligands include agents that preferentially bind to specific cell surface markers, including but not limited to E-cadherin, CD9, SSEA4, TRA-1-60, and GCTM-2. Alternatively, negative selection may be employed where undesired cell populations (e.g. lineage committed cell) are removed from the population of hESCs.

Forced Aggregation of hESCs

For the generation of differentiated cells it may b e preferable to first generate aggregated cell populations comprised of hESCs or other pluripotent cells, which are of a size which falls within an optimal range.

In order to control the size of the aggregated population harvested hESCs (or other pluripotent cells) are dissociated to become single cells through exposure to an agent which causes cell dissociation (for example 0.25% trypsin-EDTA.) Alternatively cation chelators or cadherin antibodies or the like may be utilised to effect the dissociation of the cells.

In a specific embodiment cells are then aliquoted in optimized concentrations to individual holding vessels. The optimal concentration may vary between specific cell lines or stem cell populations, but such can be determined by one skilled in the art upon reading the present descriptions and examples presented.

In a further specific embodiment of the invention, aggregation of the known concentration of hESCs may be forced by placement of the hESCs into low-attachment holding vessels which are shaped to better capture the cells, e.g., round-bottomed or conical (eg Nunc 96 well non-treated round bottom plates cat# 262162) in serum free conditions. Placement of the cells in low attachment capture plates shaped to facilitate the capture of cells (e.g., conical or round-bottomed) has been shown to reliably produce aggregated hESC populations in serum free conditions, with or without centrifugation. However, an optional centrifugation step may be desired to optimize and shorten the timeframe of the aggregation. At lower concentrations (which will produce optimal differentiation patterns for some cell lines) centrifugation will effect more efficient production of blood. It will also result in cell aggregation being effected in a time frame which is at least 2-3 days quicker than that which is effected by placement in the low-attachment holding vessels. Furthermore, if specific additives such as viscosity increasing agents (e.g., polyvinylalcohol) are added to the holding vessel prior to centrifugation only one aggregated cell population will form which may be an advantage in terms of creating an aggregate which falls within a more tightly defined size range.

Resulting aggregated cell populations will be of a defined size range since they are produced from a known concentration of cells. Such defined aggregated cell populations have an advantage in that they can preferentially drive cellular differentiation towards a specific lineage. Pertinent to the present invention, it is demonstrated herein that aggregation of cells of a specific concentration can preferentially cause the cells to differentiate into cells of the haematopoietic lineage.

In one embodiment, once hESCs or aggregated hES cell populations are obtained for culture, the aggregation techniques will take place within the culture bioreactor. It will, however, also be clear to one skilled in the art that, should aggregated cultures be desired for use in the present invention, this may be performed outside of the bioreactor system and the aggregated bodies added directly into the differentiation reactor. Where the use of controlled size aggregated bodies is used in the invention, conditions in the bioreactor system will be optimized to prevent further agglomeration of the bodies into larger, undefined bodies.

Bioreactors

The bioreactors used at the various steps of the present invention are designed to provide a culture process that can deliver medium and oxygenation at controlled concentrations and rates that mimic nutrient concentrations and rates in vivo. Bioreactors have been available commercially for many years and employ a variety of types of culture technologies. Of the different bioreactors used for mammalian cell culture, most have been designed to allow for the production of high density cultures of a single cell type and as such find use in the present invention. Typical application of these high density systems is to produce as the end-product, a conditioned medium produced by the cells. This is the case, for example, with hybridoma production of monoclonal antibodies and with packaging cell lines for viral vector production. However, these applications differ from applications where the therapeutic end-product is the harvested cells themselves as in the present invention. Once operational, bioreactors provide automatically regulated medium flow, oxygen delivery, and temperature and pH controls, and they generally allow for production of large numbers of cells. Bioreactors thus provide economies of labor and minimization of the potential for mid-process contamination, and the most sophisticated bioreactors allow for set-up, growth, selection and harvest procedures that involve minimal manual labor requirements and open processing steps. Such bioreactors optimally are designed for use with a homogeneous cell mixture or aggregated cell populations as contemplated by the present invention. Suitable bioreactors for use in the present invention include but are not limited to those described in U.S. Pat. No. 5,763,194 to Slowiaczek, et al., particularly for use as the culture bioreactor; and those described in U.S. Pat. Nos. 5,985,653 and 6,238,908 to Armstrong, et al., U.S. Pat. No. 5,512,480 to Sandstrom, et al., and U.S. Pat. Nos. 5,459,069, 5,763,266, 5,888,807 and 5,688,687 to Palsson, et al., particularly for use as the proliferation and differentiation bioreactors of the present invention.

The culture system in one aspect according to the present invention consists of a variable number of bioreactors connected to differing medium sources by sterile tubing and to one another with, in some embodiments, intervening selection apparatus. Generally, the medium is circulated through the bioreactor with the aid of a roller or pump. The bioreactors optimally include probes to measure pH, temperature, and O₂ concentration at points before and following each bioreactor(s). Information from these sensors may be monitored electronically. In addition, provision may be made for obtaining serial samples of the growth medium in order to monitor waste or electrolyte concentration, as well as proliferation and differentiation factor and nutrient concentrations. Activities of proliferation and differentiation factor samples taken from the entry and/or exit points of the bioreactors may be measured by conventional bioassays or immunoassays.

When an appropriate endpoint has been obtained in any one bioreactor (whatever the appropriate endpoint might be for that bioreactor), the cells are transferred out of the bioreactor, and most likely are fed directly into a selection apparatus or other bioreactor.

Selection apparatus are known in the art, and include FACS instrumentation and other fluorescent detection devices, immunologic methodologies, binding/immobilization assays and the like.

With any large volume cell culture, several fundamental parameters require almost constant control. Cultures must be provided with the medium that allows for, in the present invention, HESC maintenance, TA cell proliferation, TA cell differentiation—perhaps several separate differentiation cultures and conditions—as well as final cell culture/preservation. Typically the various media are delivered to the cell by a pumping mechanism in the bioreactor, feeding and exchanging the medium on a regular basis. The exchange process allows for by-products to be removed from the culture. Growing cells or tissue also requires a source of oxygen. Different cell types have different oxygen requirements, and the differing cell cultures of the present invention may have differing oxygen delivery requirements depending on the density of the culture. Accordingly, a flexible and adjustable means for providing oxygen to the cells is a desired component.

Depending on the particular culture, even distribution of the cell population and medium supply in the culture chamber can be an important process control. Such control is often achieved by use of a suspension culture design, which can be effective where cell-to-cell interactions are not important. Examples of suspension culture systems include various tank reactor designs and gas-permeable plastic bags. For cells that do not require assembly into a three-dimensional structure or require proximity to a stromal or feeder layer—such as most blood cell precursors or mature blood cells-such suspension designs may be used.

Efficient collection of the cells at the completion of the culture process is an important feature of an effective cell culture system. One approach for production of cells as a product is to culture the cells in a defined space, without physical barriers to recovery, so that simple elution of the cell product results in a manageable, concentrated volume of cells amenable to final washing in a commercial, closed system cell washer designed for the purpose. Optimally, the system would allow for addition of a pharmaceutically acceptable carrier, with or without preservative, or a cell storage compound, as well as provide efficient harvesting into appropriate sterile packaging. Optimally the harvest and packaging process may be completed without breaking the sterile barrier of the fluid path of the culture chamber.

With any cell culture procedure, a major concern is sterility. When the product cells are to be transplanted into patients-often at a time when the patient is ill or immunocompromised-absence of microorganisms is mandated. An advantage of the present cell production device over manual processes is that, as with many described bioreactor systems, once the culture is initiated, the culture chamber and the fluid pathway is maintained in a sterile, closed environment.

The Culturing Bioreactor Conditions

The bioreactor used for step 110 is a bioreactor with a “smart surface” such as that disclosed in U.S. Pat. No. 5,763,194 to Slowiaczek, et al. Herein, the phrase “smart surface” refers to a surface in a bioreactor that has been modified to comprise a ligand that binds differentially to a certain cell type. In one aspect of the present invention, the ligand is specific for hESCs, and will not bind TA cells. One such ligand is the CD30 ligand that was suggested for use for screening the cultured hESCs before transfer to the bioreactor at step 110. Because the TA cells do not bind to the smart surface, they can be removed from the culturing bioreactor and, thus, separated from the HESC culture (step 112). In the case of hESCs, the bioreactor chosen for the culturing bioreactor may have to be configured to support growth of a feeder layer in contact or in dose proximity to the hESCs. Alternatively, should a feeder-free culture be desired, the bioreactor would not have to be so configured. At this point, a separate, optional selection step (step 114) may take place in addition to the selection that takes place by virtue of the smart surface. Selection technologies have been discussed herein and are known to those skilled in the art.

In one embodiment, the hESCs are grown to a desired confluence in the reactor, and then the culture conditions altered to induce differentiation to TA cells. In this manner, the culturing bioreactor is effectively providing a method of differentiation as well as immortal pluripotent cell proliferation. The differentiation may optionally include a step of aggregation, which may take place in this reactor or in a subsequent reactor to the system.

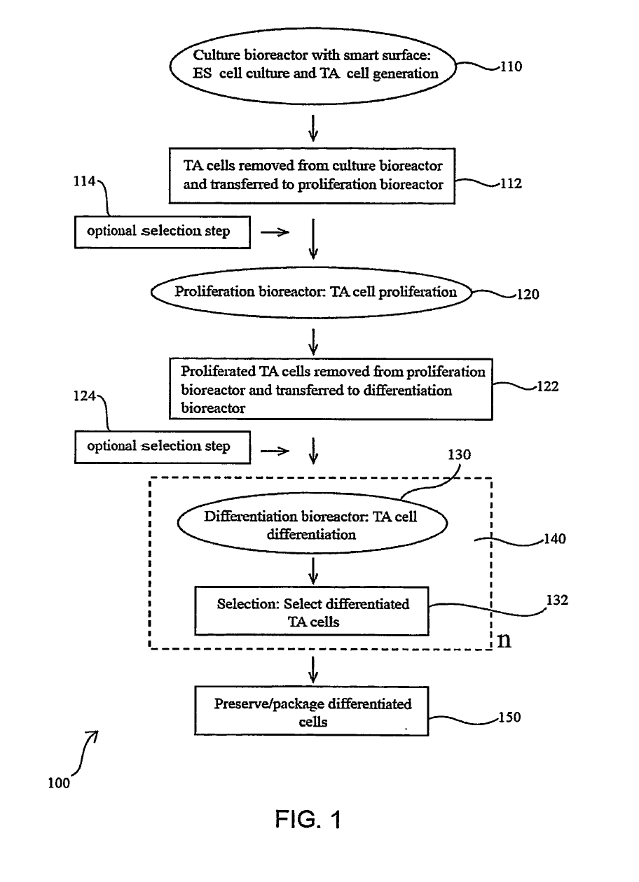

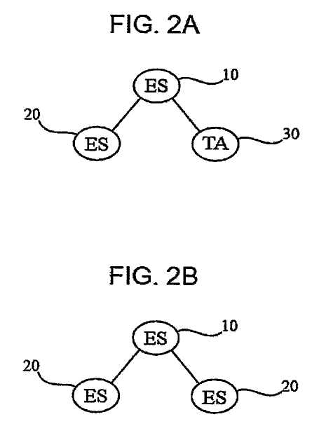

In a specific embodiment of the invention, hESCs are induced, under specific differentiating culture conditions, to undergo asymmetric division. Such a scheme is shown in FIG. 2A. In asymmetric division, an initial HESC (10) divides to produce a daughter hESC (20) and a TA cell (30). Asymmetric division leads to a steady state hESC population, generating a population of TA cells to be used for further differentiation in methods according to the present invention. FIG. 2B shows hESC amplification where the initial hESC divides to produce two daughter cells (20). In this scheme, no TA cell is generated, and the HESC population grows logarithmically. Once the HESC population reaches a desired level, the conditions are then altered to induce the differentiation of the hESC population into TA cells. Such a process of differentiation could include an aggregation step. This process (with or without aggregation) could take place in a single bioreactor, as shown in FIG. 1 at step 110, or for example when aggregated bodies are created, the hESC could be cultured in one reactor, and aggregated in a second, additional reactor (not shown). In a case where the hESC are induced to undergo asymmetric division, this could be exploited in the culturing bioreactor at step 110, where hESCs are cultured and maintained in a steady state with each hESC dividing to produce one HESC and one TA cell and where TA cells are removed from the culture bioreactor to continue through the process steps of the present invention. In each case, the ability of hESC to continually create cell populations is exploited to maximize the number of TA cells produced in the bioreactor system.

The Proliferation Reactor Conditions

The bioreactor and culture conditions used to proliferate the TA cells vary depending on the ultimate mature cell product desired. The proliferating bioreactor does not necessarily require a smart surface (though one could be employed in various aspects of the invention). Several “classic” bioreactors are known in the art and may be used, including bioreactors as described in U.S. Pat. Nos. 5,985,653 and 6,238,908 to Armstrong, et al., U.S. Pat. No. 5,512,480 to Sandstrom, et al., and U.S. Pat. Nos. 5,459,069, 5,763,266, 5,888,807 and 5,688,687 to Palsson, et al.

In general, proliferation conditions include various media. Illustrative media include Dulbecco's MEM, IMDM and RPMI-1640 that can be supplemented with a variety of different nutrients and growth factors. The media can be serum-free or supplemented with suitable amounts of autologous serum. One suitable medium is one containing IMDM, effective amounts of at least one of a peptone, a protease inhibitor and a pituitary extract and effective amounts of at least one of human serum albumin or plasma protein fraction, heparin, a reducing agent, insulin, transferrin and ethanolamine. Other suitable media formulations are the SSP media disclosed in U.S. Pat. No. 5,728,581 to Schwartz.

The proliferated TA cells from step 120 are then transferred to a third bioreactor, the differentiation bioreactor at step 122. This transfer step may or may not involve a selection/quality control step (step 124) where only TA cells of a certain phenotype are selected to go through to the next bioreactor for differentiation.

The TA cells following proliferation may be hematopoietic stem cells (HSC), hemangioblasts, uncommitted common precursors of hematopoietic cells and endothelial cells. Hemangioblasts are stable, non-transient cells that are present in both newborn infants and adults and have been isolated from cord blood. Hemangioblasts can be proliferated in a separate proliferation step or passed from the proliferation reactor to the differentiation reactor, possibly with an intervening selection step 124. If hemangioblasts are desired, selection at step 124 should enrich for cells that are Lin⁻ or CD34⁻, Cd2⁻, CD3⁻, Cd14⁻, CD16⁻, CD24⁻, CD56⁻, CD66b⁻, glycophorin A⁻, flk1⁺, CD45⁺, CXCR4⁺, and/or MDR⁺. Exemplary factors, methods, culture condition and the like for inducing differentiation of the HESC to hematopoietic precursor cells such as HSC include those disclosed in U.S. Pat. No. 6,280,718 to Kaufman et al.

Alternatively or at a more differentiated state, the proliferated TA cells may exhibit characteristics of human hematopoietic stem cells, and, as such, it would be desirable to enrich the proliferation population for cells that are CD34+, CD59+, and/or Thy-1+, CD117+ (c-kit) prior to differentiation, using for example, the method of Sutherland et al., Exp. Hematol., 20:590 (1992) or that described in U.S. Pat. No. 4,714,680 (at step 124 of FIG. 1). In addition, it may be desirable to subject the proliferated cells to negative selection to remove those cells that express lineage markers. LIN− cells lack several markers associated with lineage committed cells. Lineage committed markers include those associated with T cells (such as CD2, 3, 4 and 8), B cells (such as CD10, 19 and 20), myeloid cells (such as CD14, 15, 16 and 33), natural killer (NK) cells (such as CD2, 16 and 56), RBC (such as glycophorin A), megakaryocytes (CD41), or other markers such as CD38, CD71, and HLA-DR. Populations highly enriched in stem cells and methods for obtaining them are described in PCT/US94/09760; PCT/US94/08574 and PCT/US94/10501.

The Differentiation Bioreactor Conditions

Once the proliferated cells have been subjected to selection, they can be differentiated into a differentiated mixed cell population or a specific species of blood cell, selectively. The differentiation bioreactor may vary depending on the desired differentiated cell; however, for most applications, the differentiation bioreactor may be a “classic” bioreactor such as that suggested for the proliferating bioreactor and as described in U.S. Pat. Nos. 5,985,653 and 6,238,908 to Armstrong, et al., U.S. Pat. No. 5,512,480 to Sandstrom, et al., and U.S. Pat. Nos. 5,459,069, 5,763,266, 5,888,807 and 5,688,687 to Palsson, et al.

Differentiation conditions, such as medium components, O₂ concentration, differentiation factors, pH, temperature, etc., as well as the bioreactor employed, will vary depending on the intermediate to be differentiated and the desired differentiated cell type, but will differ primarily in the cytokine(s) used to supplement the differentiation medium. Such cytokines will be used at a concentration from about 0.1 ng/mL to about 500 ng/mL, more usually 10 ng/mL to 100 ng/mL. Suitable cytokines include but are not limited to c-kit ligand (KL) (also called steel factor), mast cell growth factor (MGF), mast cell growth and differentiation factor (MGDF), stem cell factor (SCF) and stem cell growth factor (SCGF)), vascular endothelial growth factor (VEGF), macrophage colony stimulating factor (MCSF), IL-1α, IL-2, IL-3, IL-4, IL-5, IL-6, IL-11, G-CSF, GM-CSF, MIP-1, LIF, c-mpl ligand/thrombopoietin (TPO), erythropoietin, flt3 ligand and flk2/flk3 ligand, BMP4, VEGF, and IGF2. The differentiation culture conditions will include at least two of the cytokines listed above, and may include several.

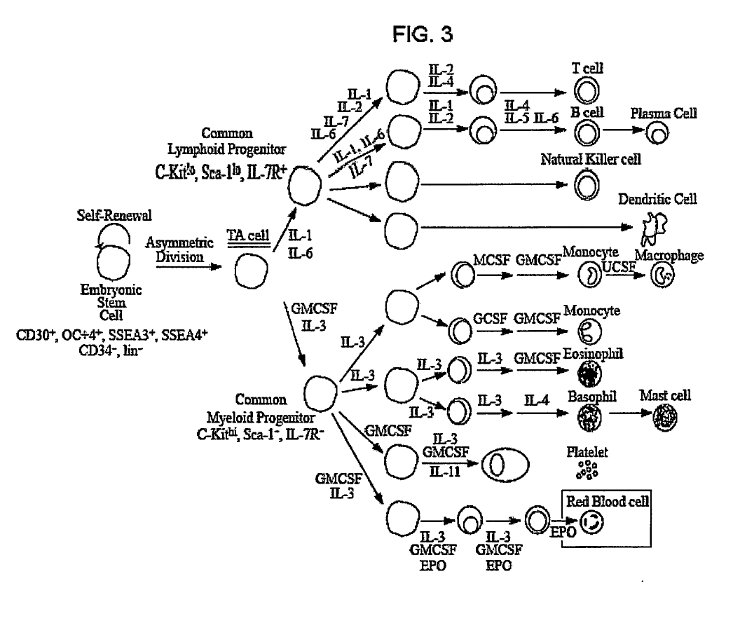

For example, if red blood cells are the desired mature blood product, at least erythropoietin will be added to the culture medium, and preferably SCGF, IL-1, IL-3, IL-6 and GMCSF all will be added to the culture medium, with erythropoietin possibly added later as a terminal differentiating factor. If platelets are the desired mature blood product, preferably SCGF and TPO and/or, IL-1, IL-3, GMSCF and IL-11 will be added to the culture medium. FIG. 3 is a diagram showing the differentiation paths of blood cells with some of the cytokines known to promote differentiation of the various cell types. For example, the path for the differentiation of T cells requires that a TA cell be differentiated with IL-1 and IL-6, followed by differentiation with IL-1, IL-2 and IL-7, followed by differentiation with IL-2 and IL4.

Alternatively to directing differentiation to a single cell type, if a single cell population is desired, the final product could be a mixed population and the cells could be separated using current cell separation techniques and procedures.

In one embodiment, the cytokines are contained in the media and replenished by media perfusion. Alternatively, the cytokines may be added separately, without media perfusion, as a concentrated solution through separate inlet ports in the differentiation bioreactor. When cytokines are added without perfusion, they will typically be added as a 10-100× solution in an amount equal to one-tenth to 1/100 of the volume in the bioreactors with fresh cytokines being added, for example, approximately every 2 to 4 days. Adding cytokines in this manner allows for progressive differentiation in the same differentiation bioreactor, as fresh concentrated cytokines also can be added separately in addition to cytokines in the perfused media.

In most aspects, suitable conditions for differentiation comprise culturing at 33 to 40° C., and usually around 37° C. The initial oxygen concentration can vary from about 1% to 20%, with approximately 5% producing oxygen partial pressures approximating the normal physiological levels in tissues. Lower oxygen concentrations have been associated with differentiation of cells to the erythrocyte lineage, so when specific cell products such as erythrocytes are desired the oxygen concentration can be kept as low as 1-5% oxygen, and preferably about 2-3% oxygen. Preferably, the cell concentration is kept at an optimum throughout differentiation.

Once differentiated, cells of the desired blood cell type can be enriched by sorting for cell surface markers. For example, T cells are known to carry the markers CD2, 3,4 and 8; B cells have CD10, 19 and 20; myeloid cells are positive for CD14, 15, 16 and 33; natural killer (“NK”) cells are positive for CD2, 16 and 56; red blood cells are positive for glycophorin A; megakaryocytes have CD41; and mast cells, eosinophils and basophils are known to have markers such as CD38, CD71, and HLA-DR.

Returning to FIG. 1, a single differentiation step is shown at step 130 and a single selection step is shown at 132 for the proliferated TA cells. However, it is contemplated that some differentiation schemes will require several, sequential differentiation and/or selection steps to achieve a homogeneous population of the desired mature cell. The possibility of such additional, sequential steps is indicated in FIG. 1 as a combined step 140, comprising a differentiation step and a selection step. Alternatively, differentiation steps can be performed sequentially, without intervening selection steps. The varying number of steps 140 is indicated by n, where n is ≧1. Thus, such a multi-step differentiation process would involve a series of TA cells, all intermediaries, displaying progressive diminution of differentiation potential and increased cell maturity as the TA cells progress through the differentiation steps. Finally, at step 160, once the desired mature cells have been produced, they are transferred to an apparatus that adds a preservation agent and/or otherwise prepares and packages the cells for storage.

Preservation

The last step shown in FIG. 1, step 160, is the preservation and packaging of differentiated cells. As described previously, the present invention envisions that to the extent possible, the system will be a dosed one. That is, delivery of the mature blood cells from the differentiation bioreactor to one or more downstream chambers or apparatus, and optimally, packaging, will take place without human intervention in a dosed, sterile system.

It may also be advantageous to preserve and package cells at a less differentiated stage, and in such an embodiment a preservative agent or appropriate conditions for preservation would take place at the stage of TA cell differentiation and/or proliferation rather than upon collection and storage of fully differentiated cells. Such cells would be stored in an incomplete differentiation state, and either used in such a state, or fully differentiated prior to clinical use

It is envisioned that in various embodiments of the present invention the downstream chambers or apparatus, will comprise one or more of the following: a chamber to wash and, if necessary, concentrate the cells or blood products; a chamber or apparatus to resuspend or perfuse the cells or blood products with a preservation or storage solution; and an apparatus to dispense and package the cells or blood products in sterile, transportable packaging. Such chambers may be separate chambers or one or more function may be performed in the same chamber. In addition, an intervening cryopreservation step (and, hence, apparatus) also may also be added.

Preservation of blood cells can be accomplished by any method known in the art. For example, general protocols for the preservation and cryopreservation of biological products such as blood cells are disclosed in U.S. Pat. Nos. 6,194,136 and 5,364,756 to Livesey, et al.; and U.S. Pat. No. 6,602,718 to Augello, et al. In addition, solutions and methods for the preservation of red blood cells are disclosed in U.S. Pat. No. 4,386,069 to Estep.

Preservation of platelets is a more difficult problem and has, until recently, been met with limited success; however, method and preservative solutions are disclosed in U.S. Pat. Nos. 5,622,867, 5,919614, and 6,211,669 to Livesey, et al., as well as recent reports regarding new methods from HyperBaric Systems, Inc. and Human Biosystems, Inc.

Packaging may be accomplished by any method or apparatus known in the art; optimally, without interruption of the sterile, closed environment. Packaging most often will involve apportioning the blood cells or blood products into sterile packaging and sealing of the packaging. An additional apparatus may be used to move the packaged product into the appropriate storage environment.

Genetic Manipulation

The blood cell products of the present invention may be modified by generating loss of function mutations by performing homologous recombination, targeted gene knockout or targeted integration in the hESCs used for culture. Such genetic manipulations may be desired particularly to delete or substitute cell surface antigens, such as histocompatability antigens or blood group antigens or to insert fluorescent or other ‘tags’ to facilitate the identification and/or isolation of differentiated progeny of hESCs.

Human Leukocyte Antigens (HLA) are part of the Major Histocompatibility Complex. The MHC comprises genes, including HLA, which are integral to normal function of the immune response. The essential role of the HLA lies in the control of self-recognition and defense against microorganisms. The HLA loci, by virtue of their extreme polymorphism, ensure that few individuals are HLA identical. HLA are recognized on all tissue cells of the body, including blood cells. Patients with intact immune systems who require multiple transfusions of whole blood, platelets or leukocyte concentrates will therefore usually develop antibodies to HLA (as well as other) antigens.

Techniques to generate recessive loss-of-function mutations in genes by are well known in the art (see, e.g., Capecchi et al., Science, 244: 1288 (1989); Hutchinson et al., Mutation Res., 299:211 (1993); Galli-Taliodoros et al., J. Immunnol. Meth., 181:1 (1995) (review); Robbins, Circ. Res., 73:3 (1993) (review); and Umans et al., J. Biol. Chem. 270:19777 (1995)). The techniques can be used to delete any gene (null mutation) including those encoding cell surface antigens, e.g., HLA histocompatibility and non-ABO blood group antigens on red blood cells, platelets, and other blood cells. For example, deletion of the beta-2-microglobulin gene will prevent expression of A, B and C antigens. In a similar manner, genes for the red blood cells antigens such as Kell, Kidd, and Duffy may, if desired, also be deleted. These techniques provide hESCs that can be differentiated into blood products that can be transfused into a patient which have a reduced ability to induce antibodies which may limit further transfusions. After transfection, the cells are commonly selected by including a second gene construct (e.g., antibiotic resistance gene) that can be utilized in a positive-negative selection process.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The described experiments were performed using two separate hESC lines. Both lines displayed an increased efficiency of hematopoietic differentiation using the aggregation techniques of the present invention. The optimum concentration for production of hematopoietic cells was found to be different for the two lines, and thus may vary between other hESC lines as well, but the optimum for each particular line can be easily determined by one skilled in the art using the described techniques.

### Example 1

Aggregation of hESC to Enhance Differentiation into Cells of the Hematopoietic Lineage

Aggregation protocols for two HESC lines were established using two methods: aggregation by gravity and aggregation facilitated by centrifugation. Both protocols utilize serum-free media and low adhesion plates designed to facilitate formation of cellular aggregates.

hESCs grown on mouse feeder cells were passaged the day before the procedure and were used in the experiments at approximately 60-80% confluency. To ensure identification of the approximate number of cells that would be present in the created aggregated bodies, the starting hESC cells were harvested, suspended in serum-free media, and the concentration of the cells determined as described below. For each flask of hESCs used, the growth medium was aspirated, and the cells washed once with PBS (Ca²⁺ and Mg²⁺ free). TVCS (0.25% trypsin/EDTA (Gibco, Life Technologies) supplemented with 2% heat inactivated chicken serum (Hunter)) was added in an amount to cover the surface of the flask (e.g., 1.5 ml per 75 cm²), and the cells incubated approximately 1 minute at room temperature, or until the feeder cells between the colonies begin to slough off. The TVCS was then aspirated, and a few mls of PBS (e.g., 3 mls per 75 cm²) added to rinse remaining TVCS from the cells.

Differentiation medium base (without growth factors) (based on the Chemically Defined Medium [CDM] described by Johansson and Wiles, MCB 15,141-151,1995 (IMDM/Ham's F12 1:1 (Gibco), BSA (Sigma), Lipids (Gibco), Ascorbic acid (Sigma), GlutaMax1(Gibco) a-MTG (Sigma), Protein free hybridoma medium (Gibco)) was added to each flask, and the hESCs dislodged from the flask by physically shaking the flask. The cells were then collected into a centrifuge tube and the cells spun at 1500 rpm, for 2 minutes at 4° C. The cells were gently resuspended in 3-5 mls of the differentiation medium base (without growth factors), and a cell count performed. A sample of the resuspended hESCs was used to determine the approximate concentration of the hESC in the resuspension solution. The number of any remaining feeder cells was excluded based primarily on the size difference between the feeder cells and the hESCs.

Additional differentiation media with growth factors added (see Table 1) was added to the cells to bring the total volume to a level resulting in the desired cell concentration. 100 μl of the cell solution was aliquoted into each well of a 96 well round-bottomed untreated low adhesion plate (Nunc, cat# 264122) to facilitate aggregation. The cells were then either returned to the incubator (37° C., 5% CO₂ in air) and allowed to aggregate by gravity (Aggregation Technique 1) or, more commonly, aggregated by spinning the plates at 1500 rpm for 4 minutes at 4° C. (Aggregation Technique 2). Following centrifugation, the wells were examined microscopically to ensure the cells were aggregated in the wells. As a control to demonstrate the facilitating effects of a round bottomed plate, in some experiments, 100 μl of the cell solution was also plated into a flat bottomed plate and incubated immediately or following a centrifugation step as described above. In all cases, the cells were incubated at 37° C., in a humidified atmosphere of 5% CO₂ in air.

### Example 2

Analysis of Differentiation of Cells to the Hematopoietic Lineage Using the Aggregation Techniques

The effect of the aggregation techniques described in Example 1 was then examined for each cell line.

Following incubation from day 0 to day 11 following aggregation, the cultured aggregates were plated into fresh, tissue culture grade 96 well flat bottomed plates to allow further expansion. Prior to plating, the plates were coated with a 0.1% gelatin solution in dH20 for at least 15 minutes, and the remaining non-attached gelatin aspirated prior to use. Additional dH20 was added to the outside wells of the coated plates to prevent desiccation of the aggregates after plating. The medium used for culture in the flat bottomed plates was a-Differentiation medium-based medium with the addition of specific blood growth factors (See Table 1).

TABLE 1
Exemplary growth factors for culture media

| | | | | working | vol for |
|---|---|---|---|---|---|
| Growth | | | | concen- | 10 mls |
| factor | supplier | stock | | tration | CDM |
| hBMP4 | R&D Systems | 100 | ng/μl | 10 ng/ml | 10 μl |
| hSCF | R&D Systems | 50 | μg/ml (50 ng/μl) | 10 ng/ml | 2 μl |
| | | | | 20 ng/ml | 4 μl |
| hVEGF | R&D Systems | 10 | μg/ml (10 ng/μl) | 5 ng/ml | 5 μl |
| hIL6 | R&D Systems | 50 | μg/ml (50 ng/μl) | 5 ng/ml | 1 μl |
| hFlt3 | R&D Systems | 25 | μg/ml (25 ng/μl) | 5 ng/ml | 2 μl |
| ligand | | | | | |
| hIGF 2 | R&D Systems | 25 | μg/ml (25 ng/μl) | 5 ng/ml | 2 μl |

For re-plating the aggregates, the medium was aspirated from the low attachment plates, taking care not to disturb the aggregated body. 100 μl of fresh differentiation medium with appropriate growth factors was added to each well, and the aggregated bodies transferred into the flat bottomed wells. The addition of the media dislodged the aggregated body to facilitate its transfer.

Following transfer, the flat bottomed plates were incubated undisturbed at 37° C., in a humidified atmosphere of 5% CO₂ in air. The plates were examined at intervals thereafter, usually between days 6 and 10 after replating, for production of cells bearing blood markers.

Blood cells were identified initially by morphology of microscopically visualized live cells and by examination of cytocentrifuge preparations stained with May Grunwald Giemsa. The cells were also analyzed by flow cytometry, using standard techniques, to identify cell surface markers, including CD45 (to identify leukocytes), Glycophorin A (to identify erythroid cells), CD34, CD117, CD116, and Flk1. Total cell survival in the wells was also measured by propidium iodide exclusion to exclude the possibility that absence of blood cells was due to a decrease in overall cell survival. Further confirmation of the hematopoietic lineages produced by the hESC differentiations could be obtained by PCR-based gene expression analysis for genes such as GATA1, GATA2, SCL, RUNX1, myeloperoxidase and globin genes.

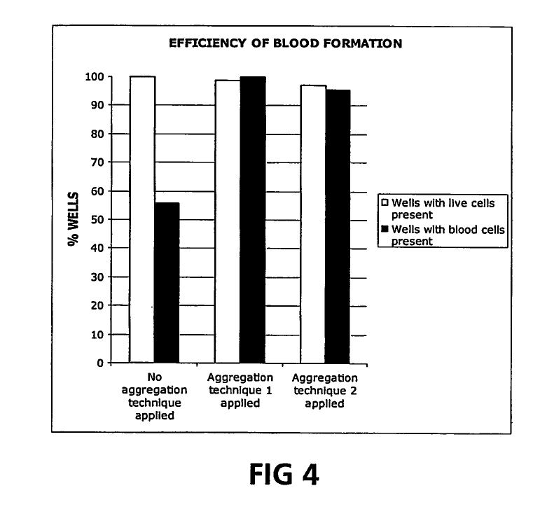

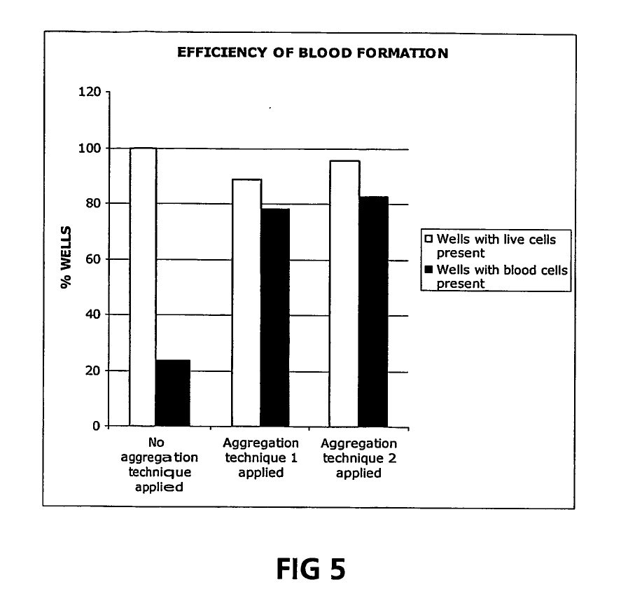

The results of the experiments for HESC line 1 (FIG. 4) and hESC line 2 (FIG. 5) both demonstrated that aggregation of the cells, either by gravity or by centrifugation, facilitated production of hematopoietic cells.

In summary, using both lines, the use of the cell aggregation techniques using serum free media and low adhesion plates shaped to facilitate aggregation (in this case round bottomed tubes) significantly increased the production of blood cells compared to use of plates that do not facilitate such aggregation.

### Example 3

Effect of Cell Density on Differentiation Efficiency

The impact of cell density on the wells was then examined using Aggregation Technique 2.

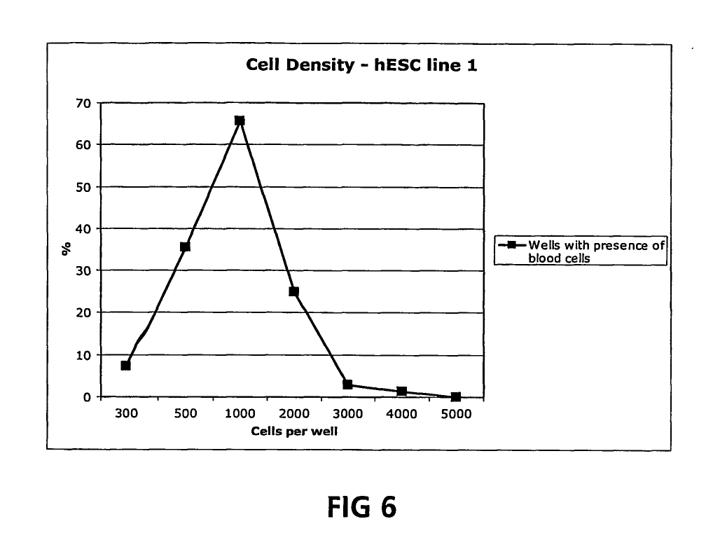

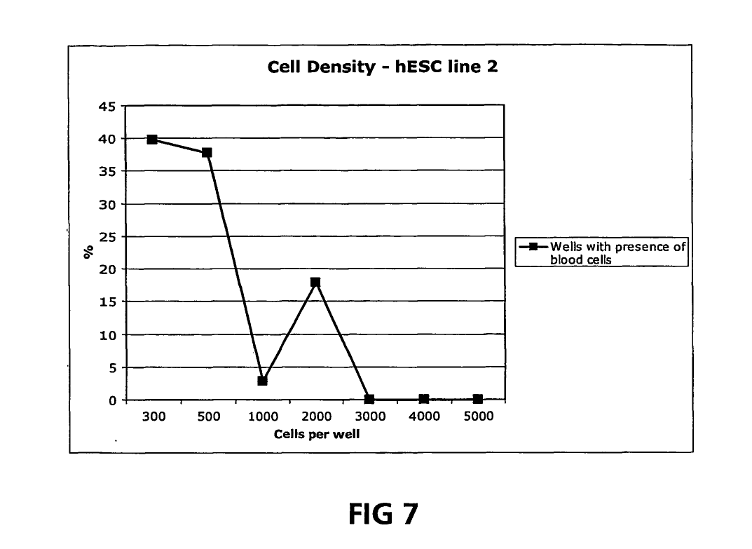

Cells were harvested in differentiation medium, and the approximate concentration of the initial cell suspension determined as described in Example 1. The concentration of the cells was then further diluted in media in varying concentrations to demonstrate differences in aggregation and differentiation efficiency. HESC populations were suspended in media to achieve the following approximate cell numbers per 100 μl: 300, 500, 1000, 2000, 3000, 4000 and 5000. The cells were aggregated according to Technique 2, i.e. by spinning the plates at 1500 rpm at 4 minutes at 4° C., and incubated as described in Example 1. The cells were incubated and replated, and numbers of wells containing blood cells was determined, as described in Example 2. Each of the cell lines examined displayed a specific optimum concentration for differentiation (See FIGS. 6 and 7) for the aggregation technique. The reason for this variation may be a result of various factors, including the sensitivity of the specific cell populations. The optimum concentration can, however, be determined for each line to be used to maximize efficiency of the techniques.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1.-11. (canceled)

12. A method, comprising the steps of:
culturing a plurality of immortal pluripotent cells in the presence of a cell culture medium under conditions which promote growth;
allowing a portion of the cells to grow and differentiate into differentiated human blood cells; and
isolating the differentiated human blood cells from the culture.

13. The method of claim 12, wherein the immortal pluripotent cells are cultured under conditions which promote asymmetric division resulting in the production of a population of daughter pluripotent cells and transient amplifying cells.

14. The method of claim 12 wherein prior to differentiation at least a portion of a plurality of immortal pluripotent cells is aggregated.

15. The method of claim 13 wherein prior to differentiation at least a portion of a plurality of immortal pluripotent cells is aggregated.

16. The method of claim 14 wherein the aggregation of at least a portion of a plurality of immortal pluripotent cells is achieved by gravity or centrifugation.

17. The method claim 12, wherein the culturing of the immortal pluripotent cells occurs in a first bioreactor, and wherein the transient amplifying cells are transferred to a second bioreactor and cultured under conditions that promote proliferation of the transient amplifying cells.

18. The method of claim 17 wherein the amplified transient amplifying cells from the second bioreactor are transferred to a third bioreactor and cultured under conditions that promote further differentiation of the transient amplifying cells.

19. The method of claim 17, wherein the first bioreactor comprises a surface which binds differentially to a specific known cell type.

20. The method claim 12, further comprising:
formulating the isolated human blood cells with an injectable carrier to provide an injectable formulation.

21. The method of claim 12, further comprising:
lysing the human blood cells; and
isolating a protein from the lysed cells.

22. The method of claim 12, wherein the immortal pluripotent cells are self renewable over a period of at least three months.

23. The method of claim 12, wherein the immortal pluripotent cells are self renewable over a period of at least six months.

24. The method of claim 12, wherein the immortal pluripotent cells are self renewable over a period of at least twelve months.

25. The method of claim 12, wherein the immortal pluripotent cells are human embryonic stem cells.

26. The method of claim 20, wherein the immortal pluripotent cells are human embryonic stem cells.

27. The method of claim 21, wherein the immortal pluripotent cells are human embryonic stem cells.

28. A method, comprising the steps of:
culturing a plurality of immortal pluripotent cells in the presence of a cell culture medium under conditions which promote growth;
allowing a portion of the cells to grow and differentiate into differentiated human blood cells;
isolating the differentiated human blood cells from the culture; and
formulating the isolated human blood cells in an injectable formulation.

29. The method claim 28, wherein the culturing of the immortal pluripotent cells occurs in a first bioreactor, and wherein the transient amplifying cells are transferred to a second bioreactor and cultured under conditions that promote proliferation of the transient amplifying cells.

30. The method of claim 29 wherein the amplified transient amplifying cells from the second bioreactor are transferred to a third bioreactor and cultured under conditions that promote further differentiation of the transient amplifying cells.

31. The method of claim 30, wherein the first bioreactor comprises a surface which binds differentially to a specific known cell type.

32. The method of claim 28, wherein the immortal pluripotent cells are human embryonic stem cells.

33. A method, comprising the steps of:
culturing a plurality of immortal pluripotent cells in the presence of a cell culture medium under conditions which promote growth;
allowing a portion of the cells to grow and differentiate into differentiated human blood cells;
isolating the differentiated human blood cells from the culture;
lysing the human blood cells; and
isolating a protein from the lysed cells.

34. The method of claim 33, wherein the immortal pluripotent cells are cultured under conditions which promote asymmetric division resulting in the production of a population of daughter pluripotent cells and transient amplifying cells.

35. The method of claim 33 wherein prior to differentiation at least a portion of a plurality of immortal pluripotent cells is aggregated.

36. The method of claim 35 wherein the aggregation of at least a portion of a plurality of immortal pluripotent cells is achieved by gravity or centrifugation.

37. The method claim 35, wherein the culturing of the immortal pluripotent cells occurs in a first bioreactor, and wherein the transient amplifying cells are transferred to a second bioreactor and cultured under conditions that promote proliferation of the transient amplifying cells.

38. The method of claim 33 wherein the amplified transient amplifying cells from the second bioreactor are transferred to a third bioreactor and cultured under conditions that promote further differentiation of the transient amplifying cells.

39. The method of claim 38, wherein the first bioreactor comprises a surface which binds differentially to a specific known cell type.

40. The method of claim 33, wherein the immortal pluripotent cells are human embryonic stem cells.
